(19) United States
(12) Patent Application Publication
(10) Pub. No.: US 2024/0038392 A1
(43) Pub. Date: Feb. 1, 2024

(54) METHOD AND SYSTEM FOR DETERMINING DIFFERENTIAL DIAGNOSIS USING A MULTI-CLASSIFIER LEARNING MODEL

(71) Applicant: Quai.MD, Ltd., Neve Ilan (IL)

(72) Inventors: Golan YONA, Tel Aviv (IL); Chen SHAPIRA, Haifa (IL); Shlomi UZIEL, Neve Ilan (IL)

(73) Assignee: Quai.MD, Ltd., Neve Ilan (IL)

(21) Appl. No.: 18/362,412

(22) Filed: Jul. 31, 2023

Related U.S. Application Data
(60) Provisional application No. 63/369,846, filed on Jul. 29, 2022.

(51) Int. Cl. G16H 50/20; G06N 3/04

(52) U.S. Cl. CPC G16H 50/20; G06N 3/04

(57) ABSTRACT

A system and method for identifying differential diagnoses using a multi-classifier disease model. The method includes constructing the multi-classifier disease model based on at least one disease profile, wherein the multi-classifier disease model is a machine learning network of at least one disease and a plurality of health variables; extracting at least one patient health variable from input patient data, wherein the input patient data indicates a patient condition; applying the multi-classifier disease model to the at least one patient health variable to determine probabilities for the at least one disease; identifying, based on the probabilities of the at least one disease, the differential diagnoses for the input patient data; and providing the differential diagnoses.

### CROSS-REFERENCE TO RELATED APPLICATIONS

This application claims the benefit of U.S. Provisional Application No. 63/369,846 filed on Jul. 29, 2022, the contents of which are hereby incorporated by reference.

### TECHNICAL FIELD

The present disclosure generally relates to clinical pathways and more particularly to determining differential diagnoses using a multi-classifier learning model.

### BACKGROUND

Clinical pathway is a tool to guide evidence-based healthcare for clinical problems with a goal of providing organized and standardized healthcare processes. The clinical pathway facilitates the decision-making process for physicians by providing a step-by-step guidance to improve patient safety and clinical efficiency. To this end, adherence to such pathways provides advantages of improving patient outcomes, reducing variations in diagnoses, costs, and more.

However, currently developed clinical pathways are isolated protocols that often focus on a single diagnosis or disease group. Such isolation and limitation do not reflect the complex, dynamic interconnections of diseases and the human body, and can cause inaccurate decisions. As an example, chest pain is a common complaint associated with up to 30 diseases where some are more frequent and easier to diagnose, but others are rare and may be more complicated to diagnose.

Using the currently developed clinical pathways, many diagnoses related to chest pain that are outside the scope of a selected isolated protocol may be unintentionally omitted and undiscovered to risk patient wellness. That is, the existing evidence-based pathways do not cover the entirety of the diagnostic space, where some, often rare, diseases may be overlooked. Currently developed clinical pathways also do not take into account co-morbidity, where the presented symptoms may be due to multiple diseases or medications. Instead, due to its isolated nature, the clinical pathways may only identify one or a short list of diseases from a large pool of diseases related to a symptom or health history.

Moreover, it has been identified that the current system of clinical pathways takes a considerable amount of time to be developed and further implemented in clinical settings. Contents of the clinical pathways require extensive research for accurate representation of relevant components such as tests, treatments, communications, and more. Once established, such carefully developed clinical pathways are often distributed in document files or in siloed applications with the expectation for physicians to abide by them. Here, the clinical pathways are rather stagnant due to effort, time, and resources required to develop. And thus, modification of clinical pathways to reflect latest clinical results or to adapt to particular communities or facilities is challenging and slow.

Current evidence-based pathways are also limited in their fidelity as only a small fraction of patient parameters, based on an “average patient,” are considered and the large variability between patients and associated patient parameters are left out in constructing the clinical pathways. Moreover, decisions within the clinical pathway are often semi-rigorous decisions (e.g., hard thresholds, utilizing a limited set of variables and/or tests) derived from limited clinical studies conducted on relatively smaller groups of patients, typically within a certain geographical region. To this end, such current clinical pathways do not appropriately represent the diverse population and thus, ill-suited for applying to all patient cases. Current pathways are also not localized to a specific area or hospital, ignoring the variability between the hospitals. The lack of principles or formalism for deriving pathways further makes it difficult for the users to adapt them to their patient's community, and patients in general. Because of this, the pathways that do exist are for the average hospital and average patient, with no room for adaptation to various impactful differences.

It should therefore be advantageous to provide a solution that would overcome the challenges noted above.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include a method for identifying differential diagnoses using a multi-classifier disease model. The method comprises: constructing the multi-classifier disease model based on at least one disease profile, wherein the multi-classifier disease model is a machine learning network of at least one disease and a plurality of health variables; extracting at least one patient health variable from input patient data, wherein the input patient data indicates a patient condition; applying the multi-classifier disease model to the at least one patient health variable to determine probabilities for the at least one disease; identifying, based on the probabilities of the at least one disease, the differential diagnoses for the input patient data; and providing the differential diagnoses.

Certain embodiments disclosed herein also include a non-transitory computer readable medium having stored thereon causing a processing circuitry to execute a process, the process comprising: constructing the multi-classifier disease model based on at least one disease profile, wherein the multi-classifier disease model is a machine learning network of at least one disease and a plurality of health variables; extracting at least one patient health variable from input patient data, wherein the input patient data indicates a patient condition; applying the multi-classifier disease model to the at least one patient health variable to determine probabilities for the at least one disease; identifying, based on the probabilities of the at least one disease, the differential diagnoses for the input patient data; and providing the differential diagnoses.

Certain embodiments disclosed herein also include a system for identifying differential diagnoses using a multi-classifier disease model. The system comprises: a processing circuitry; and a memory, the memory containing instructions that, when executed by the processing circuitry, configure the system to: construct the multi-classifier disease model based on at least one disease profile, wherein the multi-classifier disease model is a machine learning network of at least one disease and a plurality of health variables; extract at least one patient health variable from input patient data, wherein the input patient data indicates a patient condition; apply the multi-classifier disease model to the at least one patient health variable to determine probabilities for the at least one disease; identify, based on the probabilities of the at least one disease, the differential diagnoses for the input patient data; and provide the differential diagnoses.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is an example network diagram utilized to describe the various embodiments.

FIG. 2 is a flowchart illustrating a method for constructing a disease model according to an embodiment.

FIG. 3 is a schematic diagram illustrating a simplified disease model according to an example embodiment.

FIG. 4 is a flowchart illustrating a method of determining at least one differential diagnosis from a disease model according to an embodiment.

FIG. 5 is a schematic diagram of an analysis system according to an embodiment.

### DETAILED DESCRIPTION

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

The various disclosed embodiments provide a system and a method for determining differential diagnoses using a machine-learning based disease model and multi-classifier. The disease model is a network of a plurality of diseases and various health variables that are interconnected in casual relationships. The mapping between the diseases and health variables may be initially constructed using evidence-based knowledge collected through medical research, historical medical data, and more, which may be learned further and continuously modified in order to deduce more accurate and customized mapping for “non-average” patients. The disclosed embodiments provide a comprehensive and customized disease model that may be utilized to determine multiple differential diagnoses in a larger diagnostic space with improved accuracy. At least one machine learning algorithm is applied to the disease model to dynamically learn and infer probabilities of multiple differential diagnoses in the diagnostic space. It should be appreciated that the disease model is not only dynamically optimized but also expandable in that new and learned causal relationships of diseases and health variables may be easily incorporated.

According to the disclosed embodiments, the disease model may be customized for, for example but not limited to, demographics, facility, community, country, and more, in order to determine differential diagnoses for certain specific patients. Diseases are physiological disorders that manifest themselves as symptoms or other physiological signs, for example, but not limited to, levels of certain molecules in the blood or changes in the local structure of certain organs. It has been identified that observable health variables such as, but not limited to, symptoms, factors, habits, and the like, may appear in different characteristics and degrees depending on, for example, the environment, ethnicity, and more. To this end, the customized network of the disease model may more effectively learn and uncover hidden variables (i.e., diseases) that are the underlying conditions of observable health variables based on partial data of the patients. Such customized disease model would be most helpful to hospitals, where the diagnostic space may be explored more efficiently, even when presented with a patient's partial data.

Current approaches of determining diagnosis using clinical pathways are often isolated clinical pathways including a single diagnosis or few very closely related diseases based on medical knowledge. Such approaches do not reflect the complex nature and interdependencies that exist in the human body, particularly failing to represent co-morbidities. Co-morbidity implies that two or more diseases may be present at the same time in a patient, and symptoms are the aggregated effects of their presence. The disease model, of the disclosed embodiments, allows interdependencies between hidden and observable variables to represent co-morbidity and other interconnects existing in the human body. It should be appreciated that the comprehensiveness of the disclosed disease model enables more accurate reflection of a patient's health conditions based on patient data collected over time. To this end, underlying physiological conditions and less apparent disease and/or health variables may be discovered. Moreover, the adaptable and transforming nature of the model makes it possible to rapidly and closely implement changes in variable interdependencies (e.g., per community) by modifying joint probabilities within the disease model.

According to the disclosed embodiments, the multi-classification disease model enables efficient determination of differential diagnoses in a diagnostic space in conjunction with a clinical meta-pathway. A clinical meta-pathway is a comprehensive network graph designed to help explore and identify differential diagnoses of a patient. It includes a plurality of individual decision points (i.e., pathway states) which are navigated through to reach a diagnosis, if applicable. It has been identified that navigation through the clinical meta-pathway for a patient begins from a patient's chief complaint to a diagnosis and includes a few obstacles. First, the challenge may be a representation of the health space and a model of the diagnostic space that reflects proximity of related diseases. A second challenge may be establishing, when given a chief complaint, a destination or a diagnosis result, by consulting with, for example, but not limited to, various questions, examinations, tests, in order to obtain clues about the positioning of a patient in the diagnostic space.

To this end, the comprehensive disease model of the disclosed embodiments may be integrated closely to effectively determined navigation paths through the meta-pathway graph over the challenges noted above. The interrelationship and joint probabilities of health variables and underlying diseases in the disease model reflect distance between the various decision points (or pathway state) and/or diseases within the clinical meta-pathway of a patient. In particular, operative actions (or “to-do” actions) such as, but not limited to, examinations, tests, may be accurately determined to locate and guide paths through the clinical meta-pathway, and in return for effective discovery of probable differential diagnoses. It should be noted that the customized learning disease model provides accurate reflection of proximity between pathway states based on, for example but not limited to, joint probabilities learned with usage, which are otherwise not reflected in single or network of clinical pathways. It should be further noted that the multi-classification by the disclosed disease model enables efficient decision-making and navigation through meta-pathway graph by eliminating repeated analysis of health variables and/or differential diagnoses. One of ordinary skill in the art would appreciate that the learning disease model enhanced computer efficiency by reducing processing time and power, thereby conserving computing resources.

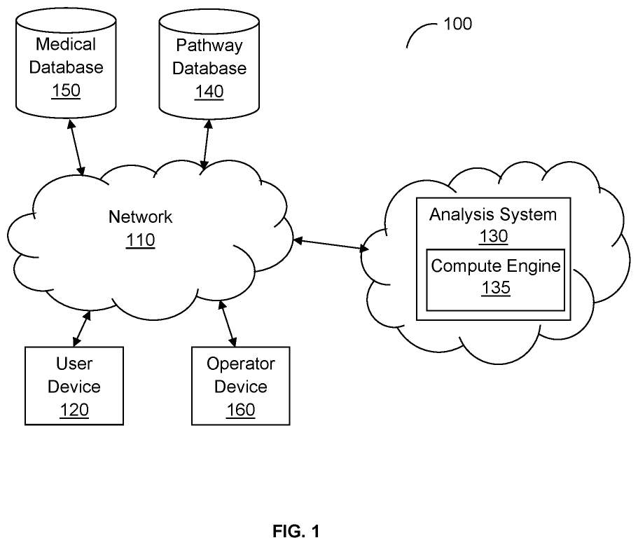

FIG. 1 shows an example network diagram 100 utilized to describe the various disclosed embodiments. In the example network diagram 100, a user device 120, an analysis system 130, a compute engine 135, a pathway database 140, a medical database 150, and an optional operator device 160 are communicatively connected via a network 110. The network 110 may be, but is not limited to, a wireless, cellular or wired network, a local area network (LAN), a wide area network (WAN), a metro area network (MAN), the Internet, the worldwide web (WWW), similar networks, and any combination thereof. The analysis system 130 may be deployed in a cloud computing platform which may be, but is not limited to, a public cloud, a private cloud, or a hybrid cloud. In some embodiments, the analysis system 130, when installed in the cloud, may operate as a software as a service (SaaS) and integrated to an. electronic health record application programming interface (EHR API).

The user device 120 and the operator device 160 may each be, but is not limited to, a personal computer, a laptop, a tablet computer, a smartphone, a wearable computing device, or any other device capable of receiving, processing, and displaying notifications. The user device 120 may be associated with a medical entity, such as a physician, that may be presented with at least one differential diagnosis (DD) from analysis of input patient data based on the multi-classifier disease model.

In an embodiment, the differential diagnosis and suggested operative action (e.g., blood test, x-ray imaging, and more) may be presented as a sub-section of clinical meta-pathway through a graphical user interface (GUI) presented on the user device 120. In a further embodiment, the GUI may be utilized to present suggestions of operative actions enabling physician involvement (e.g., by selecting) through the navigation process. It should be noted that the DDs and suggested operative actions, such as exams, tests, decisions, and the like, are customized outputs for “non-average” patients and/or geographical areas or medical facilities, obtained by applying the trained multi-classifier disease model. The construction and training of the multi-classifier disease model is discussed further hereinbelow.

The user device 120 may be utilized for authorized users to modify portions of the clinical meta-pathway based on new information from, for example but not limited to, medical research literature, facility availability, and the like, and any combination thereof. The optional operator device 160 may be associated with an operational entity, such as a billing department within the medical facility or external hospital, to provide streamline services and communications to patients.

In addition, the user device 120 may be utilized to input patient data such as, but not limited to, age, sex, ethnicity, chief complaint, test results, symptoms, and the like, through the graphical user interface (GUI) for personalized execution of the clinical meta-pathway in conjunction to the disease model. In such a scenario, input patient data from the electronic health record application programming interface (EHR API) or the GUI may be incorporated in the clinical meta-pathway as health variables for navigating through the pathway states (or decision points) and leading to a diagnosis. It should be noted that input patient data may only include partial patient data, i.e., not all information about the patient's health condition and state must be included.

The pathway database 140 may be part of the analysis system 130 or may be separate and communicatively connected via the network 110. The pathway database 140 is utilized for storing, for example, disease profiles, respective health variables, pathway states, module profiles, and more, as well as any combination thereof. In addition, the database 140 may include medical research information from, for example, medical literature, academic papers, medical portals, and more. In an embodiment, the pathway database 140 may provide medical research information to the analysis system 130 for development of the clinical meta-pathway graph. In a further embodiment, the pathway database 140 stores progress data collected from navigating through the clinical meta-pathway. In an example embodiment, the progress data includes data collected along a patient's journey over time and/or throughout the pathway. In an embodiment, the progress data may be used as feedback data to train and update the disease model.

The medical database 150 may communicate with the analysis system 130, either directly or over the network 110. The medical database 150 may be associated with the medical entity to store various medical information such as, but not limited to, patient electronic health records (EHR), facility information, equipment information, community data, and the like.

The analysis system 130 may be configured to collect medical research information and standardize the parameters including but not limited to, diagnosis, symptoms, tests, and more, to generate a plurality of disease profiles and an encompassing diagnosis space related to targeted chief complaints. It should be noted that the chief complaint may be associated with multiple diseases that are not typically categorized in a single clinical pathway protocol in conventional siloed protocols. The analysis system 130 is configured to retrieve input data from databases, such as, but not limited to, the pathway database 140, the medical database 150, as user devices (e.g., user device 120 and operator device 160), and the like to construct and update the disease profiles, and further modify the clinical meta-pathway graph. The initial disease profiles are created based on evidence-based data such as, but not limited to, medical research information, standardized parameters, and the like that are collected from databases (e.g., the pathway database 140, medical database 150, FIG. 1).

According to the disclosed embodiments, the compute engine 135 is configured to construct a disease model based on one or more disease profiles and parameters within the diagnosis space of the chief complaint. The disease model may be constructed from one or more single disease models, each created from a single disease profile. In an embodiment, the disease model is generated by the causal relationship between the health variables and the associated diseases. The health variables may be, but are not limited to, risk factors, symptoms, medications, treatments, environmental factors, co-morbidity, and a patient's medical history, among other factors which may increase the likelihood or are correlated with certain diseases.

The compute engine 135 is configured to use one or more machine learning models (i.e., the disease models) to determine a likelihood (or probability) that indicates proximity of each connection between the health variables and/or differential diagnoses (DDs) in the constructed disease model. In an embodiment, the probabilities of the constructed disease model indicate joint probability and inter-dependency between multiple variables that include co-morbidity between various hidden and observable variables, information pertaining to unknown variables that effectively increase and/or decrease the probability of related diseases, and more. In an embodiment, the constructed disease model is a comprehensive disease model including a complex network of health variables and diseases that represent the diagnosis space of interest. In a further embodiment, the probabilities of the disease model may be utilized in multi-classification to determine likelihood for multiple concurrent diseases with respect to input patient data. It should be noted that such probabilities generated in the disease model are more accurate representations of the complex human physiological conditions than conventional evidence-based data.

According to the disclosed embodiments, at least one algorithm such as a machine learning algorithm, may be applied on real-time input data such as, but not limited to, patient data, physician selection, community data, historical data, and more, to dynamically train and customize the disease model for certain group of patients. In an embodiment, the initial evidence-based disease model may be trained using a training dataset. In a further embodiment, the disease model may be trained continuously, for example, by applying a supervised machine learning algorithm, reinforcement machine learning algorithm, and the like, to improve accuracy in identifying potential DDs based on the input data. The disease model may be customized to, for example, but not limited to, a specific patient, facility, community, and the like. In an embodiment, the compute engine 135 may calculate the joint probability of various hidden (e.g., diseases) and observable variables (e.g., symptoms, factors, and the more) with respect to particular input data such as, but not limited to, historical patient data, real-time data, and more, to efficiently and accurately update the likelihood of differential diagnoses. Moreover, the connections between the health variables and the DDs may also be dynamically modified through the learning. It should be appreciated that the learning capability of the disease model creates customized disease models for certain group of patients (e.g., non-average, geographical region, etc.) for accurate and rapid convergence to DDs, thereby also conserving computing resources.

In some configurations, according to the disclosed embodiments, the comprehensive disease model may be deployed as a Bayesian Belief Network (BBN) model. The BBN model is best used when the existing variables, both hidden or observable, may either be dependent or independent from each other. The model graph structure represents the causal relationship between the diseases, which are hidden variables, and health variables such as risk factors and symptoms, which are observable or measurable variables. The terms parents and children are used to specify which variables depend on which. Risk factors are parents of diseases, while symptoms are children of diseases. Every factor that appears in a disease is linked to that disease as a causal parent, and every symptom is linked to the disease as a child. These connections, also known as edges, determine the core structure of the BBN. Each node in the BBN is associated with a conditional, often multi-dimensional, probability distribution that specifies its dependency on the values of its parents. These conditional probability (or likelihood) distributions are learned from the given patient data. The structure of the network and the parameters, which are the conditional probabilities, determine the joint probability distribution over the network's variables by taking the evidence of some other present values of variables within the network in order to infer the most likely values of the unknown variables such as the hidden disease variables, and their probability of occurrence. An algorithm such as, but not limited to, an iterative sampling procedure is used to assign values to variables in order to infer multiple unknown variables.

In an embodiment, a standard BBN model is adjusted to represent joint probability distribution of more than one class, each of varying dimension, and having the ability to be dependent or independent from each other. The adjusted BBN for the purposes of the disease model may also accommodate relationships associated with specific time intervals. It should be further appreciated that the BBN model allows development of a white box multi-classifier model that enables cross dependencies and feedback loops for continuous learning and optimization of the disease model.

In one embodiment, the disease model may include extensions based on dynamic BBNs in order to represent short-term temporal aspects and dependencies between health variables and the differential diagnoses. In yet another embodiment, the BBN-based disease model may be embedded in or encoded as an input for one or more other machine learning models such as a deep neural network (DNN) to create an advanced learning disease model. The DNN architectures may include, for example, but not limited to, recurrent neural networks (RNN), long short-term memory networks (LSTMs), Graph Neural Networks (GNN), Autoencoders, Generative Adversarial Networks (GANs), and the like.

It should be noted that the disease model is designed, constructed, and learned to differentiate between coincidental regularities and significant signals that are unlikely to occur by chance, enabling, for example, but not limited to, representation of complex scenarios of comorbidity.

According to the disclosed embodiments, the disease model may be configured as an underlying layer to the clinical meta-pathway for efficient navigation through pathway states. The analysis system 130 is configured to apply at least one algorithm such as a machine learning algorithm to navigate between pathway states of the clinical meta-pathway with improved accuracy. In an embodiment, the multi-classifier disease model is utilized to compute the probability of the next steps (i.e., different DDs at a given pathway state or decision point) and/or DDs in the clinical meta-pathway. The disease model converts the hard-threshold decisions of conventional pathways to probabilistic decisions for computations of the optimal paths for accurate and rapid convergence to a DD, if applicable. In an embodiment, the multi-classifier model determines probabilities multiple DDs and/or health variables to identify at least one DD with a probability greater than a threshold value.

The analysis system 130 utilizes the probabilities of DDs in the disease model, from the compute engine 135, to determine positions of the patient in a clinical meta-pathway, where the resolution of such a position is improved as patient information is provided and learned. In a further embodiment, the analysis system 130 suggests new inquiries or operative actions to improve prognosis based on historical collective data and the personalized patient's data. The operative action may include, for example, but not limited to, specific blood test, an X-ray, MRI, or any other medical testing that may help reveal the patient's diagnosis.

In an example embodiment, the analysis system 130, when executing the navigation algorithm, may prioritize differential diagnoses (DDs) considering multiple non-patient features such as, but not limited to, risk, prevalence, resources, time, cost and more, which may be used for identifying optimal operative actions. In such cases, explanation (i.e., notable non-patient features) may be provided to a user via a user device 120 to allow user interaction with the clinical meta-pathway. It should be appreciated that user interactions and feedback to assess and monitor navigation process may further facilitate customization of the clinical meta-pathway and/or the disease model to the user's needs.

According to the disclosed embodiments, the initially created clinical meta-pathways may be personalized (i.e., tailored automatically and dynamically in real-time for specific patients) and refined by optimizing the pathway states, based on the disease model and its generated joint probabilities that were learned from patient data. In yet another embodiment, the structure of the clinical meta-pathway graph may be optimized, for example, by suggesting new or alternative pathway states including other health variables and/or operative actions that leads to the differential diagnoses of improved accuracy. Such structure optimization may be performed by presenting the clinical meta-pathway graph to authorized medical personnel via a user device 120, for example, for auditing and reviewing.

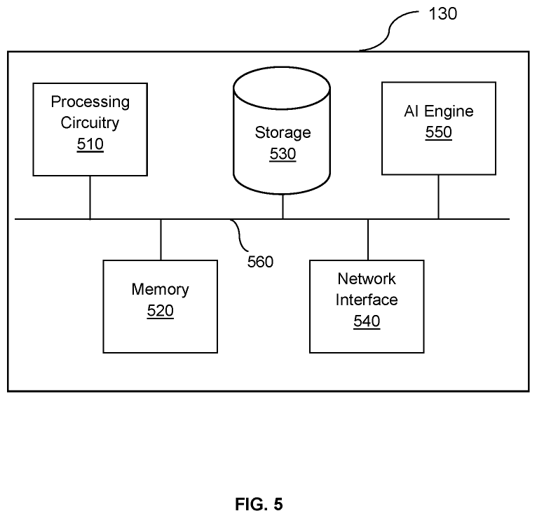

For simplicity, the compute engine 135 is shown as a component of the analysis system 130, but may be performed in a single or separate system without departing the scope of the disclosed embodiments. The system executing the described methods of the analysis system 130, the compute engine 135, or both have architectures of FIG. 5 as described below.

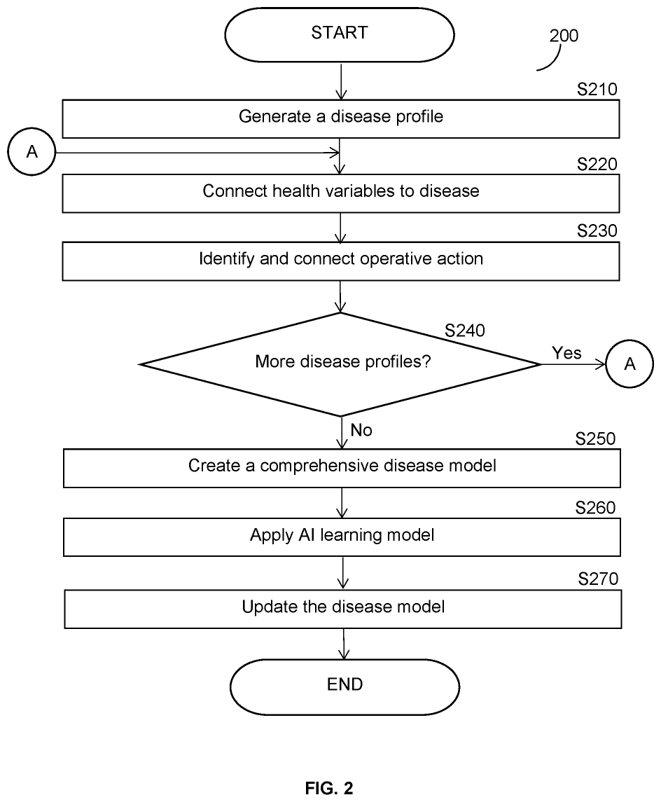

FIG. 2 is an example flowchart 200 illustrating a method for constructing a disease model according to an embodiment. The method described herein may be executed by the analysis system 130 and/or the compute engine 135, FIG. 1.

The method described herein is iteratively performed to construct a comprehensive disease model connecting the different DDs and various associated health variables to show co-dependencies and/or joint effects that are otherwise not obtained. In an embodiment, the constructed disease model may be configured and learned in a Bayesian Belief Network (BBN). In another embodiment, the constructed disease model may utilize other machine learning algorithms, such as, but not limited to, deep neural networks (of various architectures), and the like.

At S210, a disease profile is generated. The disease profile includes elements associated with the disease that are standardized and referred to as health variables. In an embodiment, the health variables may be, for example, risk factors, symptoms, tests, measurements of physiological condition of the patient, medications, treatments, genetic factors, and more. The health variable may include information such as, but not limited to, type of variable, values for each of the variables, relation of variable to disease (e.g., factor or symptom), likelihood of variable relative to disease, and the like. The disease profile is generated for each diagnosis in a differential diagnosis (DD) set that includes a plurality of diagnoses (or diseases) that are associated with a chief complaint. A chief complaint is a common symptom that is related to a large pool of diagnoses, for example, but not limited to, chest pain, dyspnea (shortness of breath), back pain, dizziness, and more. In an example embodiment, a separate clinical meta-pathway may be created for each of the different chief complaints. The plurality of disease profiles of the DD set is stored in a memory or the pathway database (e.g., the pathway database 140, FIG. 1).

As an example, a disease profile for diabetes may include excessive fatigue, high blood sugar level, blood test, family history, insulin therapy, and more. In an embodiment, health variables may be added to disease profiles to expand further to include, for example, medications, treatments, genetic factors, physiological states, and co-dependencies between diseases. In a further embodiment, the health variables and values associated may be updated with usage of the learning disease model to provide additional input data for example, but not limited to, patient data, facility data, and the like, and applying algorithms, such as, machine learning algorithms.

At S220, the diagnosis is connected to health variables to create a single disease model. The health variables such as, factors, symptoms, and the like, are connected to underlying conditions (or diseases) to show causal relationships from evidence-based knowledge of disease profiles. The terms parents and children are used to specify which variables depend on which. Risk factors are parents of diseases, while symptoms are children of diseases. Every factor that appears in a disease is linked to that disease as a causal parent, and every symptom is linked to the disease as a child. In an embodiment, the connections that make up the core structure of the model are known as edges. As an example, the underlying condition is any medical condition, such as a pulmonary embolism, liver failure, and the like, that may be shown as observable symptoms of a patient from a blood test result showing an out-of-range marker, physical symptoms such as nausea or headache, and the like. In some embodiments, the assignment for disease hidden variable may be performed randomly.

At S230, identify and connect operative action. The operative action, or “to-do” action is, for example without limitations, an examination, an imaging operation, and the like, and any combination thereof that may be performed in association to the health variables and disease. In an embodiment, the initial identification and connection of operative actions may be performed based on evidence-based knowledge and/or historical data that are available from databases (e.g., the pathway database 140 and the medical database 150, FIG. 1).

At S240, a check is performed whether the DD set includes more disease profiles. If so, the operation returns to S220, otherwise, operation continues to S250. Operations S220 to S230 is performed for each diagnosis (i.e., disease profile) in the DD set to create and connect a plurality of single disease models for the plurality of diseases associated with the chief complaint.

At S250, the plurality of single disease models is connected to generate a disease model. The disease model is a comprehensive network of single disease models linked together through the health variables. In an embodiment, each node of the disease model may be independent or dependent on each other. In an embodiment, the disease model is created to show statistical dependencies between variables, such as between health variables and diseases, and characterizes their joint probability distribution. Such models allow integration of prior evidence-based knowledge, which may be used to guide model construction where the disease profiles and evidence-based pathways are utilized. The probability distributions may be utilized to identify the more probable diagnoses over others. Moreover, such probability distributions are analyzed to suggest the next operative actions upon receiving patient input data. In an embodiment, the diagnosis and operative actions may be determined from the disease model, for example, by comparing patient values of a health variable to a predetermined threshold value. In an example embodiment, the diagnosis and/or operative action may be connected and constructed when the patient value is greater than the predetermined threshold value of the health variable. In an embodiment, the predetermined threshold value may be updated from evidence-based values as feedback data is received through learning by applying the disease model.

In an embodiment, the combined disease model network also exhibits causal relationships between diseases and various health variables to show parent and children relationships. It should be noted that such a network provides multi-dimensional probability distribution of variables and their values to diseases. It should also be appreciated that the network nature of the disease model allows the network to be expandable to include additional single disease models to be integrated into the disease model without disrupting the overall construction of the network.

At S260, an AI model is applied to the generated disease model. The AI model is an algorithm, such as a machine learning algorithm, trained to determine the joint probabilities of health variables and the diseases, using historical patient data. That is, the likelihood of each of the diseases may be determined and dynamically modified through learning from new data collected. In an example embodiment, the new data used for learning may include, for example but not limited to, historical patient data, current patient data, community data, facility data, medical knowledge, and the like, and any combination thereof.

In an embodiment, the disease model may be developed as a Bayesian Belief Network (BBN) that represents interconnections of hidden and observable variables as well as their conditional dependencies. It should be appreciated that, in such scenarios and the like, the disease model network may be accessed and visible to enable the decision making explainable for a user utilizing the disease model and/or the associated clinical meta-pathway.

At S270, the disease model is updated based on learning through the AI model. In an embodiment, the disease model may be dynamically modified to reflect new medical discoveries, patient groups, community, facility, and the like, and any combination thereof. In an embodiment, the likelihood (or probabilities) of at least one differential diagnoses in the disease model may be modified based on feedback data. The feedback data may be collected from, for example, but not limited to, a user (or physician) selection, patient data, historical data, patients' progress data, and the like, and more. In further embodiment, the predetermined threshold value associated with the disease and health variable connection may be updated according to data collected and applied to the learning disease model.

As an example, an initial threshold level for creatinine is set to 52, and immediate cardiology evaluation is recommended when creatinine level is greater than 52. In the same example, the threshold level for creatinine may be reduced to 48 for a patient in community A, based on learning and updating of the disease model to better serve community A. To this end, the learning disease model enables more accurate and efficient analyses of input patient data that may be customized for, as examples without limitations, patients, groups of patients, communities, facilities, demographics, and the like, and any combination thereof.

Moreover, the disease model is configured as a multi-classifier disease model that efficiently and accurately identifies the differential diagnoses (DDs). Based on the comprehensive network, the multi-classifier disease model determines probabilities for one or more DDs based on partial patient data. The interconnected network eliminates redundant processing and determination of probabilities to improve computer efficiency, conserve computing resources of processing power, time, and memory. It should be understood by a person of ordinary skill in the art that manual analysis interconnected plurality of health variables and differential diagnoses are not only challenging but lack implications of dependencies to realize such network and probabilistic discovery.

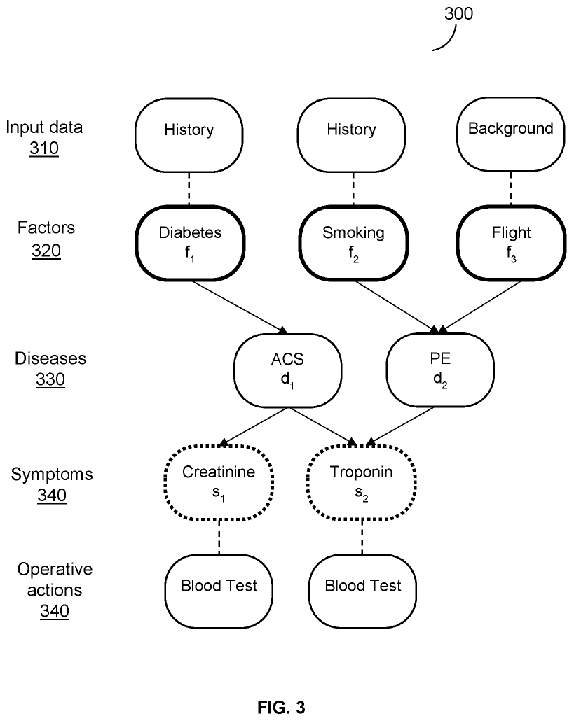

FIG. 3 is an example schematic diagram 300 illustrating a simplified disease model according to an example embodiment. The example disease model 300 shows the causal relationship between two hidden variables (i.e., diseases 330) and associated observable variables such as, but not limited to, various factors 320, symptoms 340, and the like. In the example embodiment, the simplified disease model is created for acute coronary syndrome (ACS) and pulmonary embolism (PE).

The network of the simplified disease model 300 represents the causal relations between the factors 320 (e.g., smoking), the diseases 330 (e.g., pulmonary embolism), which are the underlying conditions being estimated by the disease model 300, and the symptoms 340 (e.g., higher level of troponin). In an embodiment, the skeleton structure of the disease model 300 is based on the evidence-based knowledge of the disease profiles. The disease model 300 shows interdependencies in the diseases 330, ACS and PE, sharing common factors 320 and/or symptoms 340.

The disease model 300 may be implemented to determine operative actions 340 based on input data 310, for example but not limited to, patient historical data, patient background data, community data, and the like, and any combination thereof. The factors 320 may be extracted from the input data 310 and used in the multi-classifier disease model to generate probabilities of different DDs. Applying the disease model may be repeated for each extracted health variable that are associated with the DD set of interest. It should be noted that the probabilities of DDs with respect to the health variable indicate proximities and may be updated with continuous learning from feedback data. Each health variable has an associated operative action 340 that may be used to reveal a related disease 330 based on prior disease knowledge.

In an embodiment, the disease model 300 including probabilities (or likelihood) of each disease 330 are utilized to determine the appropriate operative actions 340 (e.g., blood test), and ultimately the related diseases 330 for a differential diagnosis. It should be noted that probabilities for the diseases 330 and operative actions 340 are initially determined based on evidence-based knowledge, but the conditional probabilities are learned over time based on, for example, but not limited to, patient data, community data, physician feedback, and the like, and any combination thereof. The disease model 300 may infer the optimal next operative action 340 based on the estimated probabilities of the corresponding health variable.

In a further embodiment, the structure of the disease model 300 is applied to navigate through pathway states of the clinical meta-pathway to identify the optimal operative action 340 for a next step and/or next pathway state. It should be appreciated that the disease model 300 is created to implement inter-dependencies and joint probabilities of multiple hidden variables and observable variables to show co-morbidity. It should be further appreciated that the constructed disease model 300 is a learning model in which probabilities and classification within the model may be progressively learned and improved with usages. The disease model may be effective customized for certain groups of patients, for example, in a community, in a geographical region, and the like, and any combination thereof. To this end, the learning disease model enables rapid decision and navigation through the meta-clinical pathway while considering multiple health variables and DDs and further enables discovery of less known DDs that are otherwise difficult to obtain using conventional or manual methods of determination.

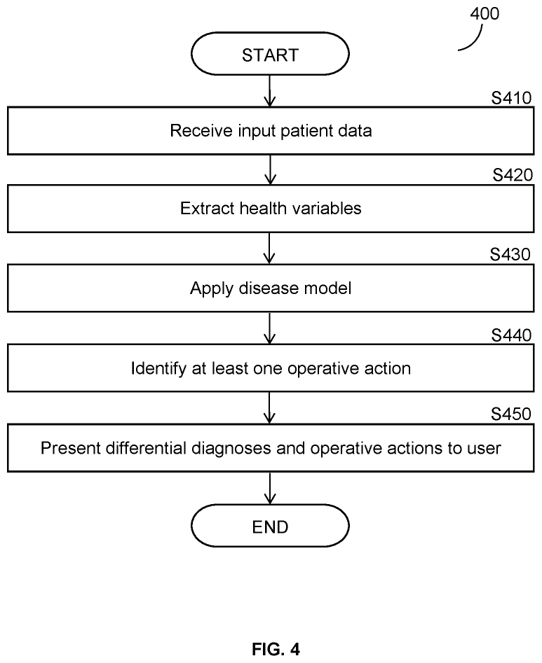

FIG. 4 is an example flowchart 400 illustrating a method of identifying at least one differential diagnosis based on a multi-classification disease model according to an embodiment. The method described herein may be executed by the analysis system 130, FIG. 1.

At S410, input patient data are received. The input patient data includes patient history and background, for example but not limited to, demographics, prior diseases, epigenetics, genetic background, habitat, family history, and the like, and any combination thereof. The patient history and background may be received from the patient as historical patient data or from a database which stores patient historical data, or in real-time through the Electronic Medical Records (EMR) system. In an example embodiment, a BBN of the disease model, based on the patient history and background, that shows a causal relationship between various health variables and diseases may be utilized.

At S420, health variables are extracted from the input patient data. Health variables include observable variables such as influencers, risk factors, symptoms, and the like that are causally related to underlying conditions (or diseases). In an example embodiment, the influencers and/or risk factors, such as, diabetes, smoking, drinking, and the like, are extracted from the patient data that are causally related to underlying conditions, for example, kidney failure.

At S430, the multi-classifier disease model is applied to the patient data and the extracted health variables. As noted above, the disease model is a machine learning network of observable and hidden variables that are interconnected based on causal relationships. In an embodiment, probabilities (or likelihoods) of one or more differential diagnoses may be determined by applying the disease model. It should be noted that the probabilities determined reflect joint probabilities of different health variables and diagnoses that may be interdependent in the constructed disease model. In an embodiment, at least one differential diagnosis may be identified based on a specific probability greater than a predetermined score. In another embodiment, the at least one differential diagnosis may be identified by a probability that is greater than probabilities determined for other differential diagnoses. The multi-classifier disease model enables dynamic inferences of probabilities for multiple DDs simultaneously for efficient identification of DDs.

At S440, at least one operative action is identified. The operative action is a “to-do” action such as, but not limited to, a blood test, an examination, an ultrasound imaging, and the like, to be performed. In an embodiment, an optimal operative action may be selected which provides the missing component to differentiate between the probable differential diagnoses determined in S430. In further embodiment, the optimal operative action may be identified by applying additional input data such as, but not limited to, facility data, community data, and the like, which may indicate availability of operative actions and prioritize certain operative actions over others.

At S450, at least one differential diagnosis and the at least one operative action is presented to a user via a user device (e.g., the user device 120, FIG. 1). In an embodiment, the user is, for example, a physician looking to determine at least one diagnosis of the patient. The at least one differential diagnosis may be presented with their respective probabilities determined in the disease model (S430). In such a scenario, the user may be presented with a suggestion of at least one differential diagnosis and one or more operative actions that help distinguish such differential diagnoses. In an embodiment, the example flowchart 400 to identify DDs may be iteratively repeated when, for example, the presented at least one DD and the at least one operative action is not selected by the user. The operation continues to S410 and performed through S450 until a decision is confirmed based on a predetermined rule and/or user input.

In an embodiment, such information on differential diagnosis and operative action may be presented as a sub-section of the clinical meta-pathway via a graphical user interface (GUI) to help navigation through the complex pathway. In further embodiment, the suggestions may be determined and presented dynamically and in real-time as patient input data are received. It should be noted that the disease model provides probabilities and optimal suggestions that are learned and thus, enables the shortest and most efficient navigation through the diagnostic space. To this end, the clinical meta-pathway is improved upon to represent a more efficient navigational path from the chief complaint to a diagnosis.

In an embodiment, feedback data with respect to the presented differential diagnoses and the operative actions taken by users may be collected for optimization of the disease model and the compute engine. The feedback data may include, for example but not limited to, historical patient data, selection of operative action, EHR data, and the like, and any combination thereof that are collected via, for example without limitations, a user device, operator device, (e.g., the user device 120 and operative device 160, FIG. 1) and more.

In an embodiment, the clinical meta-pathway may provide a comprehensive network of clinical pathways that integrate a wide range of potential diagnoses in a single clinical meta-pathway. In an embodiment, navigation through the clinical meta-pathway graph may allow objective determination of clinical decisions and/or actions and potentially lead to at least one diagnosis associated with the chief complaint. Determination of differential diagnoses (DDs) based on evidence-based clinical pathways and users (e.g., physicians) may often be subjective and based on feeling or limited knowledge of the known knowledge. However, the probabilistic network of the learning disease model provides objective rules-based decision and discovery of DDs. In addition, the disease model that reflects the large diagnostic space may reveal unknown or rare DDs that are not yet apparent in evidence-based methods.

In an embodiment, the generated plurality of pathway states that compose the clinical meta-pathway graph may be connected to one or more other pathway states. In an embodiment, a module profile may be created for each of the pathway states to include, for example but not limited to, a set of module DD set, a set of module variables, module function, a list of next steps (i.e., next pathway states connected to current pathway states), and the like. Elements in the module profile may all be associated with the potential diagnoses applicable and included in the set of module DDs. The set of module variables may be identified from the variable comparison table and include respective costs for the module variables. The module function is a distribution function over a set of module variables that may be utilized to determine a relative probability of each diagnosis in the module DD set as a probable differential diagnosis (DD) for the current pathway states.

As an example, a specific diagnosis, myocarditis, may have a likelihood of 99% on a variable “troponin” when the troponin value exceeds a predetermined threshold, in a certain pathway state The value of troponin may be above that threshold for a specific patient at the pathway state to identify myocarditis as a probable DD for the current pathway state. In an embodiment, the module function that indicates probable diagnoses may be utilized to identify clusters of diagnoses in generating pathway states.

In addition, the list of next steps may include a plurality of next pathway states that are connected to the current pathway state. In an embodiment, the next pathway state may be selected by identifying the next variable that needs to be determined (and the associated operation), based on module functions of the module variables. The next pathway state may be determined in order to rule out and/or rule in at least one diagnosis in the module DD set. In an embodiment, the pathway state may be an assessment state that helps to resolve the diagnosis most effectively. For example, the assessment state may request a blood iron level reading from the patient's EHR in order to identify and determine the next steps of the pathway state.

The patient-specific AI model may be utilized to determine values for health variables and select certain variables over others to create a patient-specific clinical meta-pathway graph. That is, navigation through the constructed clinical meta-pathway may be customized for the specific patient to provide efficient and accurate clinical operations. In an embodiment, the AI model may be initially created based on evidence-based medical knowledge.

As an example, based on the trained AI model, the clinical meta-pathway of a diabetes patient may be led to one cluster of the DD set over another cluster or may be directed to avoid a certain “to-do” action that may be unclear for effective rule in or rule out of the diagnoses.

FIG. 5 is an example schematic diagram of an analysis system 130 according to an embodiment. The analysis system 130 includes a processing circuitry 510 coupled to a memory 520, a storage 530, a network interface 540, and an artificial intelligence (AI) engine 550. In an embodiment, the components of the analysis system 130 may be communicatively connected via a bus 560. In an embodiment, the analysis system 130 and the compute engine 135 may be configured in a single component, device, server, and the like. Further, the compute engine 135 may be realized by the architecture including the elements shown in FIG. 5.

The processing circuitry 510 may be realized as one or more hardware logic components and circuits. For example, and without limitation, illustrative types of hardware logic components that can be used include field programmable gate arrays (FPGAs), application-specific integrated circuits (ASICs), Application-specific standard products (ASSPs), system-on-a-chip systems (SOCs), graphics processing units (GPUs), tensor processing units (TPUs), general-purpose microprocessors, microcontrollers, digital signal processors (DSPs), and the like, or any other hardware logic components that can perform calculations or other manipulations of information.

The memory 520 may be volatile (e.g., random access memory, etc.), non-volatile (e.g., read only memory, flash memory, etc.), or a combination thereof.

In one configuration, software for implementing one or more embodiments disclosed herein may be stored in the storage 530. In another configuration, the memory 520 is configured to store such software. Software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed by the processing circuitry 510, cause the processing circuitry 510 to perform the various processes described herein.

The storage 530 may be magnetic storage, optical storage, and the like, and may be realized, for example, as flash memory or other memory technology, compact disk-read only memory (CD-ROM), Digital Versatile Disks (DVDs), or any other medium which can be used to store the desired information, including cloud storage.

The network interface 540 allows the analysis system 130 to communicate with, for example, the network 110.

The AI engine 550 may be realized as one or more hardware logic components and circuits, including graphics processing units (GPUs), tensor processing units (TPUs), neural processing units, vision processing units (VPU), reconfigurable field-programmable gate arrays (FPGA), and the like. The AI engine 550 is configured to perform, for example, machine learning based on input data such as patient data, selection data at pathway state, feedback data, and more, received over the network 110.

It should be understood that the embodiments described herein are not limited to the specific architecture illustrated in FIG. 5, and other architectures may be equally used without departing from the scope of the disclosed embodiments.

The various embodiments disclosed herein can be implemented as hardware, firmware, software, or any combination thereof. Moreover, the software is preferably implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be understood that any reference to an element herein using a designation such as “first,” “second,” and so forth does not generally limit the quantity or order of those elements. Rather, these designations are generally used herein as a convenient method of distinguishing between two or more elements or instances of an element. Thus, a reference to first and second elements does not mean that only two elements may be employed there or that the first element must precede the second element in some manner. Also, unless stated otherwise, a set of elements comprises one or more elements.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; 2A; 2B; 2C; 3A; A and B in combination; B and C in combination; A and C in combination; A, B, and C in combination; 2A and C in combination; A, 3B, and 2C in combination; and the like.

What is claimed is:

1. A method for identifying differential diagnoses using a multi-classifier disease model, comprising:
constructing the multi-classifier disease model based on at least one disease profile, wherein the multi-classifier disease model is a machine learning network of at least one disease and a plurality of health variables;
extracting at least one patient health variable from input patient data, wherein the input patient data indicates a patient condition;
applying the multi-classifier disease model to the at least one patient health variable to determine probabilities for the at least one disease;
identifying, based on the probabilities of the at least one disease, the differential diagnoses for the input patient data; and
providing the differential diagnoses.

2. The method of claim 1, further comprising:
iteratively updating the multi-classifier disease model based on feedback data collected from applying the multi-classifier disease model.

3. The method of claim 1, further comprising:
generating the at least one disease profile, wherein each of the at least one disease profile has the plurality of health variables associated with the disease of the at least one disease; and
connecting the plurality of health variables with the at least one disease.

4. The method of claim 3, wherein the at least one disease profile is generated for the disease in a differential diagnoses set associated with a chief complaint, wherein the chief complaint is a common symptom related to a plurality of differential diagnoses.

5. The method of claim 1, wherein a health variable of the plurality of health variable is any one of: risk factor, symptom, test, measurement of physiological condition, medication, treatment, and genetic factor.

6. The method of claim 1, wherein the multi-classifier disease model is a Bayesian Belief Network.

7. The method of claim 1, wherein the identified differential diagnoses have the probabilities greater than a threshold value.

8. The method of claim 1, further comprising:
connecting an operative action to a health variable of the plurality of health variables based on historical data.

9. The method of claim 1, further comprising:
determining an operative action based on the differential diagnoses, wherein the operative action is a “to-do” action associated with at least one of the differential diagnoses.

10. A non-transitory computer readable medium having stored thereon instructions for causing a processing circuitry to execute a process, the process comprising:
constructing a multi-classifier disease model based on at least one disease profile, wherein the multi-classifier disease model is a machine learning network of at least one disease and a plurality of health variables;
extracting at least one patient health variable from input patient data, wherein the input patient data indicates a patient condition;
applying the multi-classifier disease model to the at least one patient health variable to determine probabilities for the at least one disease;
identifying, based on the probabilities of the at least one disease, differential diagnoses for the input patient data; and
providing the differential diagnoses.

11. A system for identifying differential diagnoses using a multi-classifier disease model, comprising:
a processing circuitry; and
a memory, the memory containing instructions that, when executed by the processing circuitry, configure the system to:
construct the multi-classifier disease model based on at least one disease profile, wherein the multi-classifier disease model is a machine learning network of at least one disease and a plurality of health variables;
extract at least one patient health variable from input patient data, wherein the input patient data indicates a patient condition;
apply the multi-classifier disease model to the at least one patient health variable to determine probabilities for the at least one disease;
identify, based on the probabilities of the at least one disease, the differential diagnoses for the input patient data; and
provide the differential diagnoses.

12. The system of claim 11, wherein the system is further configured to:
iteratively update the multi-classifier disease model based on feedback data collected from applying the multi-classifier disease model.

13. The system of claim 11, wherein the system is further configured to:
generate the at least one disease profile, wherein each of the at least one disease profile has the plurality of health variables associated with the disease of the at least one disease; and
connect the plurality of health variables with the at least one disease.

14. The system of claim 13, wherein the at least one disease profile is generated for the disease in a differential diagnoses set associated with a chief complaint, wherein the chief complaint is a common symptom related to a plurality of differential diagnoses.

15. The system of claim 11, wherein a health variable of the plurality of health variable is any one of: risk factor, symptom, test, measurement of physiological condition, medication, treatment, and genetic factor.

16. The system of claim 11, wherein the multi-classifier disease model is a Bayesian Belief Network.

17. The system of claim 11, wherein the identified differential diagnoses have the probabilities greater than a threshold value.

18. The system of claim 11, wherein the system is further configured to:
connect an operative action to a health variable of the plurality of health variables based on historical data.

19. The system of claim 11, wherein the system is further configured to:
determine an operative action based on the differential diagnoses, wherein the operative action is a “to-do” action associated with at least one of the differential diagnoses.
